(19) United States
(12) Patent Application Publication
(10) Pub. No.: US 2015/0224144 A1
(43) Pub. Date: Aug. 13, 2015

(54) ANTI-COMPLEMENT THERAPY COMPOSITIONS AND METHODS FOR PRESERVING STORED BLOOD

(71) Applicant: THE UAB RESEARCH FOUNDATION, Birmingham, AL (US)

(72) Inventors: Scott Barnum, Birmingham, AL (US); Rakesh P. Patel, Hoover, AL (US); Jordan Welnberg, Memphis, TN (US)

(21) Appl. No.: 14/422,871

(22) PCT Filed: Aug. 30, 2013
(86) PCT No.: PCT/US2013/057573

§ 371 Date: Feb. 20, 2015

Related U.S. Application Data
(60) Provisional application No. 61/695,451, filed on Aug. 31, 2012.

(51) Int. Cl. A61K 35/18; C12N 5/078; A61K 45/06

(52) U.S. Cl. CPC A61K 35/18; A61K 45/06; C12N 5/0641; C12N 2501/998

(57) ABSTRACT

Provided herein is a composition comprising transfusable red blood cells having improved storage capability, wherein a C9 inhibitor has been administered to the composition and thereby the composition has a reduced amount of red blood cell lysis as compared to a control. It is a surprising finding of the present invention that addition of a C9 inhibitor to a blood sample decreases the amount of red cell lysis in the sample over time. Accordingly, the present invention provides red blood cell compositions that can be stored for greater lengths of time before use, i.e., transfusion, and/or that have a reduced amount of storage lesion.

### CROSS REFERENCE TO RELATED APPLICATIONS

This application claims the priority benefit of U.S. Provisional Patent Application Ser. No. 61/695,451 filed Aug. 31, 2012.

### STATEMENT REGARDING FEDERALLY SPONSORED RESEARCH OR DEVELOPMENT

This invention was made with Government support under Grant No. HL095468 awarded by the National Institutes of Health National Heart, Lung, and Blood Institute and Grant No. AI083820 awarded by the National Institutes of Health National Institute of Neurological Disorders and Stroke. The Government has certain rights in this invention.

### BACKGROUND OF THE INVENTION

1) Field of the Invention

The field of the invention is red blood cell storage.

2) Description of Related Art

It is well established that structural and biochemical changes occur during the storage of red blood cells (RBCs) for transfusion (Weinberg et al. Transfusion 2011, 51: 867-873). Changes in older RBCs potentiate adverse patient outcomes post-transfusion, including increased susceptibility to infection, post-surgical complications, and death (Weinberg et al. Transfusion 2011, 51: 867-873; Gauvin et al. Transfusion 2010, 50: 1902-1913). Although the pathologic relevance of the storage lesion has been questioned (Middelburg et al. Transus. Med. Rev. 2013, 27: 36-42), adverse outcomes are common in trauma patients (Weinberg et al. Transfusion 2011, 51: 867-873). In this clinical setting, the so-called RBC storage lesion may deliver a “second injury” in which host inflammatory mechanisms contribute to storage lesion toxicity (Kim-Shapiro et al. Transfusion 2011, 844-851).

Complement is a potent inflammatory mediator functioning to protect the host against infection, in part, by lysing pathogens via the membrane attack complex (MAC) (Esser, A. F. Toxicology 1994, 87: 229-247). Complement is activated during leukoreduction of whole blood, generating activation fragments which may contribute to storage lesion toxicity (Seghatchian, G. Transfusion Apheresis Sci. 2003, 29: 105-117.). The complement cascade includes complement factors: C1 (C1q, C1r, and C1s), C2 (C2a and C2b), C3 (C3a and C3b), C4 (C4a, C4b), C5 (C5a, C5b), C6, C7, C8, C9, factor B, factor D, and factor P. Complement factors C6, C7, C8 and multiple C9 molecules assemble at the end of the complement cascade to form the membrane attack complex (MAC). This complex binds to the membrane of a target cell, such as a red blood cell, and forms a pore in the membrane. The result of such pore formation is lysis of the target cell.

More specifically, there are two main complement cascade pathways, the classical complement pathway and the alternative complement pathway. The classical pathway is triggered by activation of the C1-complex. The C1-complex is composed of 1 molecule of C1q, 2 molecules of C1r and 2 molecules of C1s, or C1qr2s2. Activation of the C1-complex begins either when C1q binds to IgM or IgG complexed with antigens or when C1q binds directly to the surface of the target cell or other appropriate activating surface. Such binding leads to conformational changes in the C1 q molecule, which leads to the activation of two C1r molecules.

Since C1r is a serine protease, activation of C1r leads to cleavage of C1 s (another serine protease). The C1r2s2 component then cleaves C4 and C2 to produce C4a, C4b, C2a, and C2b. C4b and C2b bind to form the classical pathway C3-convertase (C4b2b complex), which cleaves C3 into C3a and C3b; C3b later joins with C4b2b (the C3 convertase) to make C5 convertase (C4b2b3b complex).

The alternative pathway does not rely on target-binding antibodies like the classical pathway. In the alternative pathway, spontaneous C3 hydrolysis occurs due to the breakdown of the internal thioester bond. The resulting C3(H₂O) molecule binds to factor B which then is cleaved by factor D. The resulting C3(H₂O)Bb complex serves as an initiating C3 convertase for the alternative pathway, cleaving C3 to C3a and C3b. The resulting C3b moiety reacts with a hydroxyl or amino group of a molecule on the surface of a cell or pathogen covalently attaching to the surface. This C3b molecule binds to factor B to form C3bB and in the presence of factor D is cleaved into Ba and Bb. Bb remains associated with C3b to form C3bBb, which is the alternative pathway C3 convertase. The C3bBb complex of the alternative pathway is stabilized by binding oligomers of factor P. The stabilized C3 convertase, C3bBbP, then acts enzymatically to cleave more C3, some of which becomes covalently attached to the same surface as C3b. This newly bound C3b recruits more B, D and P molecules and greatly amplifies the complement activation. Once the alternative C3 convertase enzyme is formed on a pathogen or cell surface, it may bind covalently to another C3b, to form C3bBbC3bP, the C5 convertase.

Once C5 convertase is formed in either the classical or alternative complement cascade pathway, the C5 convertase cleaves C5 to C5a and C5b. The C5b molecule then recruits and assembles C6, C7, C8 and multiple C9 molecules to form the membrane attack complex (MAC). This creates a hole or pore in the membrane that can kill or damage the pathogen or cell.

### BRIEF DESCRIPTION OF THE DRAWINGS

FIG. 1 is a graph showing changes in the level of fluid-phase C5b-9 (MAC) in human blood, stored for one to six weeks, as determined by ELISA.

FIG. 2 (A-F) are graphs showing changes in the levels of C3a, C5a, Bb, iC3b, C4d and C5b-9 (MAC) in leukoreduced RBC units, stored for one to six weeks, as determined by ELISA.

FIG. 3 is a graph showing a reduction in cell-free hemoglobin in red blood cells treated with purified rabbit anti-C9 IgG (100 μg) and then stored for a total of 42 days.

### DETAILED DESCRIPTION OF THE INVENTION

Since storage of red blood cells (RBC) in blood banks for up to 42 days is a mainstay for transfusion therapeutics, there is a need for compositions and methods that reduce storage lesion toxicity. The present disclosure indicates that activation of the complement cascade and the subsequent formation of the membrane attack complex (MAC) are key to inducing adverse changes in red blood cells (RBC) during storage. It further indicates that administration of anti-complement therapies that block the formation of the MAC during RBC storage will delay and/or prevent changes in the RBC fragility and lysis. These therapies may include but are not limited to antibodies to the terminal complement pathway proteins (C9) and other agents that block or interfere with the formation of the MAC. The following definitions are used in the specification and claims to describe the present invention.

### DEFINITIONS

As also used herein, the singular form “a,” “an,” and “the” include plural references unless the context clearly dictates otherwise. For example, the term “a cell” includes a plurality of cells, including mixtures thereof

The term “antibody” is used herein in the broadest sense, and specifically covers monoclonal antibodies (including full-length monoclonal antibodies), polyclonal antibodies, and multispecific antibodies (e.g., bispecific antibodies). Antibodies (Abs) and immunoglobulins (Igs) are glycoproteins having the same structural characteristics. While antibodies exhibit binding specificity to a specific target, immunoglobulins include both antibodies and other antibody-like molecules which lack target specificity. Native antibodies and immunoglobulins are usually heterotetrameric glycoproteins of about 150,000 daltons, composed of two identical light (L) chains and two identical heavy (H) chains. Each heavy chain has at one end a variable domain (V_{H}) followed by a number of constant domains. Each light chain has a variable domain at one end (V_{L}) and a constant domain at its other end.

The term “antibody fragment” refers to a portion of a full-length antibody, generally the target binding or variable region. Examples of antibody fragments include Fab, Fab′, F(ab′)₂ and Fv fragments. The phrase “functional fragment or analog” of an antibody is a compound having qualitative biological activity in common with a full-length antibody. For example, a functional fragment or analog of an anti-IgE antibody is one which can bind to an IgE immunoglobulin in such a manner so as to prevent or substantially reduce the ability of such molecule from having the ability to bind to the high affinity receptor, FcεRI. As used herein, “functional fragment” with respect to antibodies refers to Fv, F(ab) and F(ab′)₂ fragments. An “Fv” fragment is the minimum antibody fragment which contains a complete target recognition and binding site. This region consists of a dimer of one heavy and one light chain variable domain in a tight, non-covalent association (V_{H}-V_{L} dimer). It is in this configuration that the three CDRs of each variable domain interact to define a target binding site on the surface of the V_{H}-V_{L} dimer. Collectively, the six CDRs confer target binding specificity to the antibody. However, even a single variable domain (or half of an Fv comprising only three CDRs specific for a target) has the ability to recognize and bind target, although at a lower affinity than the entire binding site. “Single-chain Fv” or “sFv” antibody fragments comprise the V_{H} and V_{L} domains of an antibody, wherein these domains are present in a single polypeptide chain. Generally, the Fv polypeptide further comprises a polypeptide linker between the V_{H} and V_{L} domains, which enables the sFv to form the desired structure for target binding.

The Fab fragment contains the constant domain of the light chain and the first constant domain (CH1) of the heavy chain. Fab′ fragments differ from Fab fragments by the addition of a few residues at the carboxyl terminus of the heavy chain CH1 domain including one or more cysteines from the antibody hinge region. F(ab′) fragments are produced by cleavage of the disulfide bond at the hinge cysteines of the F(ab′)₂ pepsin digestion product. Additional chemical couplings of antibody fragments are known to those of ordinary skill in the art.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single target site. Furthermore, in contrast to conventional (polyclonal) antibody preparations, which typically include different antibodies directed against different determinants (epitopes), each monoclonal antibody is directed against a single determinant on the target. In addition to their specificity, monoclonal antibodies are advantageous in that they may be synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies for use with the present invention may be isolated from phage antibody libraries using the well-known techniques. The parent monoclonal antibodies to be used in accordance with the present invention may be made by the hybridoma method first described by Kohler and Milstein, Nature 256, 495 (1975), or may be made by recombinant methods.

“Humanized” forms of non-human (e.g. murine) antibodies are chimeric immunoglobulins, immunoglobulin chains or fragments thereof (such as Fv, Fab, Fab′, F(ab′)₂ or other target-binding subsequences of antibodies), which contain minimal sequence derived from non-human immunoglobulin. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody may also comprise at least a portion of an immunoglobulin constant region (Fc), typically that of a human immunoglobulin template chosen.
The term “C9” includes a polypeptide sequence denoted “CO9_HUMAN, P02748” in the UniProtKB/Swiss-Prot database and any homologs thereof.

The term “C9 inhibitor” includes all molecules that bind to a complement cascade component C9 and thereby inhibit lysis of a target cell such as a red blood cell. In some embodiments, a C9 inhibitor is a C9 antibody. In other or further embodiments, the C9 antibody is specific for a C9 complement cascade component.

The terms “complement membrane attack complex” or “complement MAC” refer to a complex or association of C5b, C6, C7, C8 and multiple C9 complement molecules on a surface of a target cell such as a red blood cell. “Complement membrane attack complex (MAC) activity” refers herein to pore formation in and/or lysis of the target cell caused by the MAC.

A “composition” is intended to mean a combination of active agent and another compound or composition, inert (for example, a detectable agent or label) or active, such as an anticoagulant.

As used herein, the term “comprising” is intended to mean that the compositions and methods include the recited elements, but not excluding others. “Consisting essentially of” when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

A “control” is an alternative subject or sample used in an experiment for comparison purposes. A control can be “positive” or “negative.” In some embodiments, a control is a red blood cell sample to which a C9 inhibitor has not been added or administered.

An “effective amount” is an amount sufficient to effect beneficial or desired results. An effective amount can be administered in one or more administrations, applications or dosages.

“Mammal” for purposes of administration refers to any animal classified as a mammal, including human, domestic and farm animals, nonhuman primates, and zoo, sports, or pet animals, such as dogs, horses, cats, cows, etc.

The term “storage lesion” refers herein to a set of biochemical and/or biomechanical changes which occur during storage of red blood cells and that reduce red blood cell viability and/or the ability of the stored blood cells to adequately oxygenate tissues following transfusion.

The term “subject” is defined herein to include animals such as mammals, including, but not limited to, primates (e.g., humans), cows, sheep, goats, horses, dogs, cats, rabbits, rats, mice and the like. In some embodiments, the subject is a human.

As used herein, the term “transfusable” refers to a composition that meets or exceeds accepted medical standards for a substance to be used in the transfusion of a subject, and in particular, standards for blood compositions to be used in the transfusion of a subject.

### DESCRIPTION

Provided herein is a composition comprising transfusable red blood cells having improved storage capability, wherein a C9 inhibitor has been administered to the composition and thereby the composition has a reduced amount of red blood cell lysis as compared to a control. It is a surprising finding of the present invention that addition of a C9 inhibitor to a blood sample decreases the amount of red cell lysis in the sample over time. Accordingly, the present invention provides red blood cell compositions that can be stored for greater lengths of time before use, i.e., transfusion, and/or that have a reduced amount of storage lesion.

The C9 inhibitor used in the present invention can be any molecule that binds to a complement cascade component C9 and thereby inhibit lysis of a target cell such as a red blood cell. In some embodiments, a C9 inhibitor is a C9 antibody or a fragment thereof. The C9 antibody can be any type of antibody fragment, and in some embodiments, the C9 antibody is an F(ab′)₂ fragment. In other or further embodiments, the C9 antibody is humanized. In other or further embodiments, the C9 antibody is specific for a C9 complement cascade component, and more particularly, specific for an epitope on C9 that is not available for binding once C9 complexes to form a MAC. Included herein is a C9 antibody having a specificity of mAb #A223 (Quidel Corp., San Diego, Calif., USA).

The red blood cells described herein may be in the form of a blood sample obtained previously from a subject. The red blood cells may be within a whole blood sample, may be obtained from whole blood and subsequent red blood cell separation, or may be obtained directly from a subject via red blood cell apheresis. Further, the transfusable red blood cell composition may include other additives including, but not limited to, anti-coagulants, preservatives, and nutrient additives. Some examples of anti-coagulants are citrate-phosphate dextrose (CPD), heparin, and EDTA. Some examples of nutrient additives are saline-adenine-glucose (SAG), SAG-mannitol (SAGM), AS-₁ Adsol (Baxter), AS-₃ Nutricel (Pall Medical), AS-₅ Optisol (Terumo), MAP, and PAGGSM (MacroPharma). In some embodiments, the transfusable red blood cell composition is a packed red blood cell composition (pRBC). In some embodiments, the transfusable red blood cell composition is leukoreduced and/or irradiated. The term “leukoreduced” refers to a composition which has undergone removal of all or most of the white blood cells from the composition.

The present invention provides a transfusable red blood cell composition that can be stored for greater lengths of time before use, i.e., transfusion. In some embodiments, transfusable red blood cell composition is not frozen and is stored ex vivo at a temperature between approximately 1 and 8° C., 5 and 8° C., or 6 and 7° C. In some embodiments, the transfusable red blood cell composition may be stored ex vivo for greater than 42 days, and more particularly, between 42 and 50 days, between 45 and 50 days, between 42 and 60 days, between 50 and 60 days, greater than 45 days, greater than 50 days, greater than 60 days, or greater than 70 days.

The composition comprising transfusable red blood cells described herein has a reduced amount of red blood cell lysis and/or an increase in red blood cell viability as compared to a control. In some embodiments, the composition has a reduced amount of red blood cell storage lesion and/or transfusion toxicity as compared to a control. The term “storage lesion” refers herein to a set of biochemical and/or biomechanical changes which occur during storage of red blood cells and that reduce red blood cell viability and/or the ability of the stored blood cells to adequately oxygenate tissues following transfusion. In some embodiments, these characteristics may be attributed to a reduced amount of complement membrane attack complex (MAC) activity in the transfusable red blood cell composition as compared to a control.

Accordingly, provided herein is a method of making a transfusable red blood cell composition having improved storage capability, comprising providing a red blood cell sample and adding a C9 inhibitor to the sample. The C9 inhibitor used in the method of making a transfusable red blood cell composition can be any molecule that binds to a complement cascade component C9 and thereby inhibits lysis of a target cell such as a red blood cell. In some embodiments of the method, the C9 inhibitor is a C9 antibody or a fragment thereof. The C9 antibody can be any type of antibody fragment, and in some embodiments, the C9 antibody is an F(ab′)₂ fragment. In other or further embodiments of the method, the C9 antibody is humanized. In other or further embodiments of the method, the C9 antibody is specific for a C9 complement cascade component, and more particularly, specific for an epitope on C9 that is not available for binding once C9 complexes to form a MAC. The C9 inhibitor can be added in one or multiple administrations.

Since the method of making a transfusable red blood cell composition provided herein results in a composition that has a reduced amount of red blood cell lysis and/or an increase in red blood cell viability as compared to a control, in some embodiments, the composition is stored unfrozen for greater than 42 days. In some embodiments of the method, the transfusable red blood cell composition is not frozen and is stored ex vivo at a temperature between approximately 1 and 8° C., 5 and 8° C., or 6 and 7° C. In some embodiments of the method, the transfusable red blood cell composition may be stored ex vivo for greater than 42 days, and more particularly, between 42 and 50 days, between 45 and 50 days, between 42 and 60 days, between 50 and 60 days, greater than 45 days, greater than 50 days, greater than 60 days, or greater than 70 days.

The method of making a transfusable red blood cell composition may further comprise leukoreducing the composition and/or adding an anticoagulant and/or a nutrient additive to the composition. Some examples of anti-coagulants are citrate-phosphate dextrose (CPD), heparin, and EDTA. Some examples of nutrient additives are saline-adenine-glucose (SAG), SAG-mannitol (SAGM), AS-₁ Adsol (Baxter), AS-₃ Nutricel (Pall Medical), AS-₅ Optisol (Terumo), MAP, and PAGGSM (MacroPharma).

Also provided herein is a method of improved storage of a transfusable red blood cell composition, comprising 1) providing the composition comprising transfusable red blood cells, wherein a C9 inhibitor has been added to the composition and thereby the composition has a reduced amount of red blood cell lysis as compared to a control, and 2) storing the composition at a temperature between approximately 1 and 8° C. In this method, improved storage may result in the ability to store the composition for a longer period of time prior to use, reduced storage lesion in the composition, and/or improved transfusion quality of the composition. In some embodiments of this method, the composition is stored unfrozen for greater than 42 days.

In some embodiments of the improved storage method, the transfusable red blood cell composition is not frozen and is stored ex vivo at a temperature between approximately 1 and 8° C., 5 and 8° C., or 6 and 7° C. In some embodiments of the method, the transfusable red blood cell composition may be stored ex vivo for greater than 42 days, and more particularly, between 42 and 50 days, between 45 and 50 days, between 42 and 60 days, between 50 and 60 days, greater than 45 days, greater than 50 days, greater than 60 days, or greater than 70 days.

It should be understood that the transfusable red blood cell composition that is stored according to the method provided herein may or may not contain a C9 inhibitor during storage. In some embodiments, at least some of the C9 inhibitor is removed upon making the transfusable red blood cell composition and prior to storage. In some embodiments, 100%, 99%, 98%, 97%, 96%, 95%, 90% or 85% of the C9 inhibitor is removed from the transfusable red blood cell composition prior to or during storage. The C9 inhibitor can be removed from the transfusable red blood cell composition via any method known to those of skill in the art including, but not limited to, affinity chromatography and size exclusion chromatography. The present invention also includes a method of adding a C9 inhibitor, allowing the inhibitor to bind to a C9, and removing the C9 inhibitor from a transfusable red blood cell composition multiple times during storage of the composition.

The transfusable red blood cell composition that is stored according the method provided herein can be any transfusable red blood cell composition described above or below. In some embodiments, the transfusable red blood cell composition may include other additives including, but not limited to, anti-coagulants, preservatives, and nutrient additives. Some examples of anti-coagulants are citrate-phosphate dextrose (CPD), heparin, and EDTA. Some examples of nutrient additives are saline-adenine-glucose (SAG), SAG-mannitol (SAGM), AS-₁ Adsol (Baxter), AS-₃ Nutricel (Pall Medical), AS-₅ Optisol (Terumo), MAP, and PAGGSM (MacroPharma). The red blood cells used to make the transfusable red blood cell composition may be in the form of a blood sample obtained previously from a subject. The red blood cells may be within a whole blood sample, may be obtained from whole blood and subsequent red blood cell separation, or obtained directly from a subject via red blood cell apheresis. In some embodiments, the transfusable red blood cell composition is a packed red blood cell composition (pRBC). In some embodiments, the transfusable red blood cell composition is leukoreduced and/or irradiated.

Further provided herein are methods for reducing toxicity of stored red blood cells and/or reducing red blood cell storage lesion in a red blood cell sample. Accordingly, provided herein is a method of reducing toxicity of a stored red blood cell composition comprising providing a red blood cell sample, adding a C9 inhibitor to the sample, storing the sample, and thereby creating a stored red blood cell composition having reduced toxicity. Also provided herein is a method of reducing storage lesion in a red blood cell sample providing a red blood cell sample, adding a C9 inhibitor to the sample, storing the sample, and thereby creating a stored red blood cell composition having reduced storage lesion. In some embodiments, the red blood cell sample is stored ex vivo at refrigerated temperatures beyond 42 days.

It should be understood that the foregoing relates to preferred embodiments and that numerous changes may be made therein without departing from the scope of the disclosure. The following examples further illustrate these embodiments, which examples are not to be construed in any way as imposing limitations upon the scope thereof. On the contrary, it is to be clearly understood that resort may be had to various other embodiments, modifications, and equivalents thereof, which, after reading the description herein, may suggest themselves to those skilled in the art without departing from the spirit of the disclosure and/or the scope of the appended claims. Further, all publications, patents and patent applications cited herein are hereby incorporated by reference in their entirety for all purposes.

### EXAMPLES

### Example 1

### Identification of Complement Activation During Red Blood Cell Storage

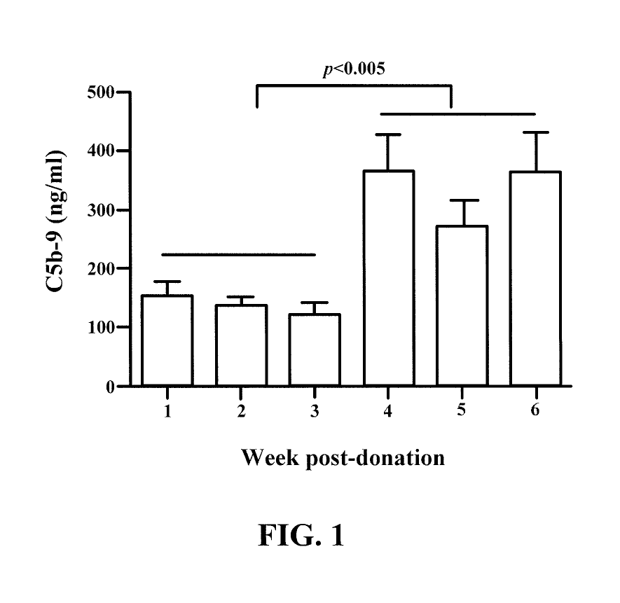

For some studies, “pigtails” of human blood, stored for one through six weeks, were assayed using the C5b-9 (MAC) ELISA (Quidel Corp.) for changes in the level of fluid-phase MAC. In the first three weeks post-donation, MAC levels were low and not significantly different. At week four, fluid-phase MAC levels increased significantly (p<0.005, Students t-test) and stayed elevated through six weeks post donation. RBC stored according to UAB blood banking conditions for one to six weeks were assayed for C5b-9 by solid-phase ELISA. Data shown in FIG. 1 are the mean +/−SEM of 7 to 12 samples per time point. The studies shown in FIG. 1 suggest that treatment of packed RBCs with an antibody to C9 to inhibit formation of the MAC may prevent insertion of fluid-phase MAC into RBC membranes and reduce or prevent hemolysis.

In other studies, a cross-sectional analysis was performed of aliquots of leukoreduced RBC units, stored for one to six weeks, for the levels of C3a, C5a, Bb, iC3b, C4d and C5b-9 (MAC) by ELISA. Aliquots were aseptically obtained from leukoreduced units in additive solution (types, O, A and B) prior to transfusion at the University of Alabama at Birmingham or the University of Tennessee Health Science Center. Study approval was obtained from the institutional review boards at both facilities. Supernatants were assessed for levels of C3a, C5a, Bb, iC3b, C4d and C5b-9 (MAC) by ELISA (Quidel, Corp., San Diego) and statistical analysis was performed using Prism 5 (Graphpad Software, San Diego, Calif.). A p value of less than 0.05 was considered significant.

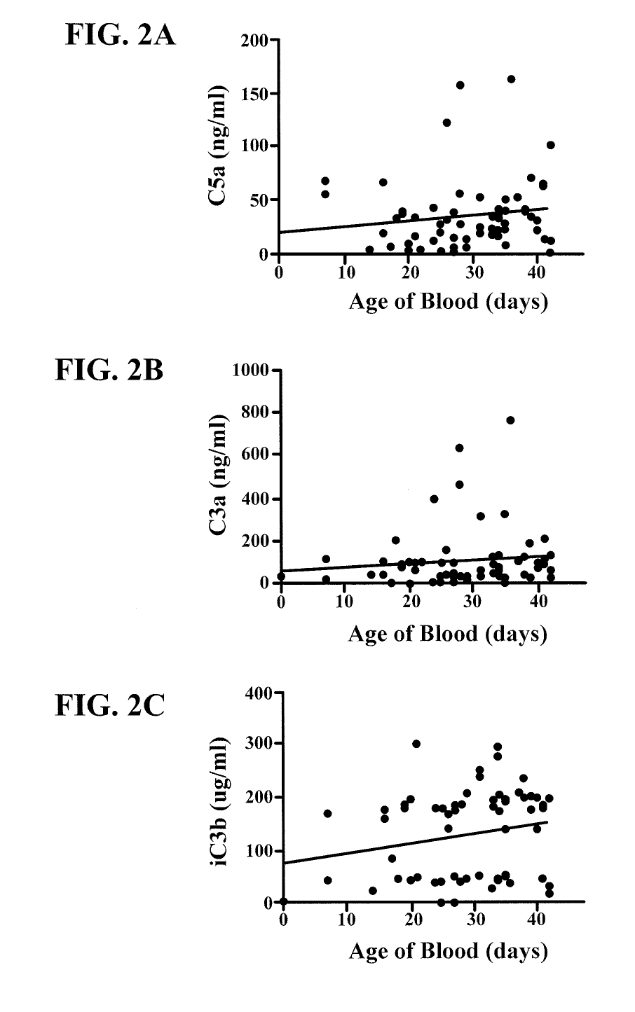

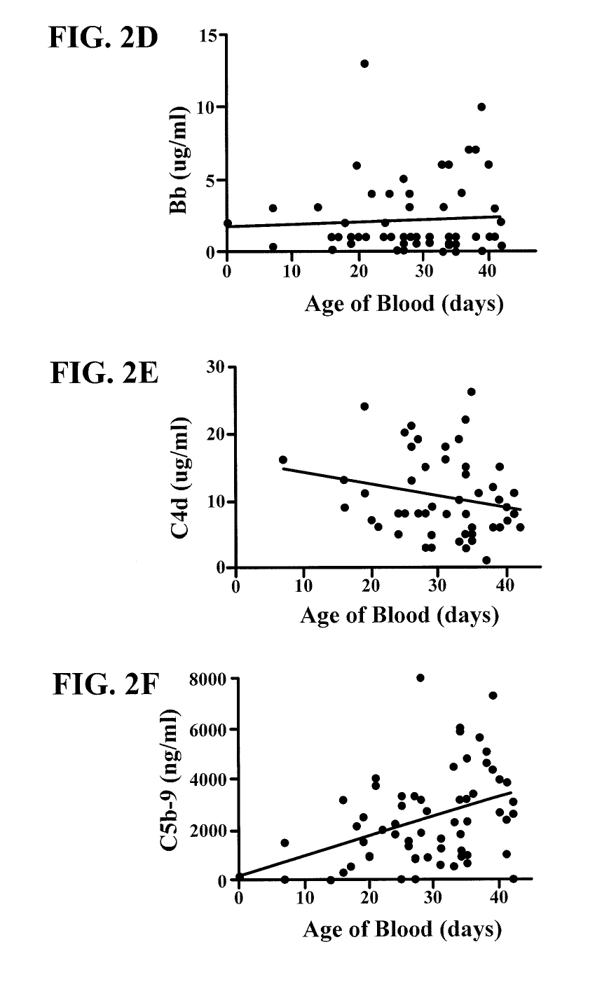

FIG. 2 shows that the levels of C5a, C3a, iC3b and Bb did not increase in RBC units stored from one to six weeks at 2-6° C. (FIG. 2A-D). C4d levels correlated negatively with storage time, but not significantly (Pearson's correlation=−0.24, p=0.09) (FIG. 2E). In contrast, a significant, time-dependent increase in C5b-9 levels was observed (Pearson's correlation=0.15, p=0.001) (FIG. 2F).

### Example 2

### Addition of Anti-C9 Antibody Markedly Prevents Accumulation of Cell-Free Hemoglobin in Stored RBC

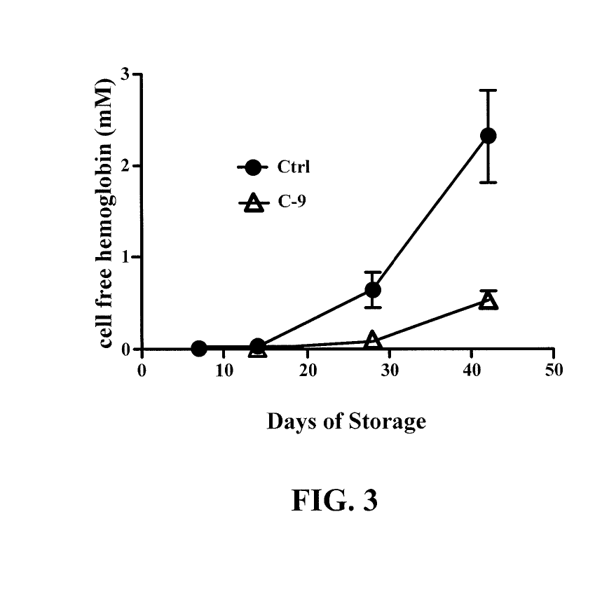

Red blood cells stored for seven days according to UAB blood banking conditions were either untreated or treated with purified rabbit anti-C9 IgG (100 μg) and then stored for a total of 42 days. At days 7, 14, 28 and 42, aliquots were removed and assayed for cell-free hemoglobin. Data shown in FIG. 3 are the mean +/−SEM for 3 samples per group. These data suggest that inhibition of C9 via anti-C9 antibody or through other means of blocking formation of the MAC may significantly reduce RBC hemolysis and extend the storage time of RBCs for transfusion. This would increase the usable blood supply for transfusion and may also reduce transfusion-related toxicity.

1. A composition comprising transfusable red blood cells having improved storage capability, wherein a C9 inhibitor has been administered to the composition and thereby the composition has a reduced amount of red blood cell lysis as compared to a control.

2. The composition of claim 1, wherein the C9 inhibitor is a C9 antibody.

3. The composition of claim 1, wherein the composition is capable of being stored ex vivo and unfrozen for greater than 42 days.

4. The composition of claim 3, wherein the composition is stored ex vivo at a temperature between approximately 1 and 8° C.

5. The composition of claim 4, wherein the temperature is approximately 6° C.

6. The composition of claim 1, wherein the composition is leukoreduced.

7. A method of making a transfusable red blood cell composition having improved storage capability, comprising providing a red blood cell sample and adding a C9 inhibitor to the sample.

8. The method of claim 7, wherein the C9 inhibitor is a C9 antibody.

9. The method of claim 7, further comprising storing the composition unfrozen for greater than 42 days.

10. The method of claim 9, wherein the composition is stored at a temperature between approximately 1 and 8° C.

11. The method of claim 10, wherein the temperature is approximately 6° C.

12. The method of claim 7, further comprising leukoreducing the composition.

13. A method of improved storage of a transfusable red blood cell composition, comprising
a. providing the composition comprising transfusable red blood cells, wherein a C9 inhibitor has been added to the composition and thereby the composition has a reduced amount of red blood cell lysis as compared to a control, and
b. storing the composition at a temperature between approximately 1 and 8° C.

14. The method of claim 13, wherein the composition is stored for greater than 42 days.

15. The method of claim 13, wherein the temperature is approximately 6° C.

16. The method of claim 13, wherein the composition is leukoreduced.

17. The method of claim 13, wherein the composition further comprises an anticoagulant and/or a nutrient additive.
